(19) United States
(12) Patent Application Publication
(10) Pub. No.: US 2004/0260400 A1
(43) Pub. Date: Dec. 23, 2004

(54) Device for fitting a uterine suspension prosthesis for the treatment of uterine prolapse

(76) Inventor: Edgard Cornier, Paris (FR)

Correspondence Address: BAKER & DANIELS, 111 E. WAYNE STREET, SUITE 800, FORT WAYNE, IN, 46802

(21) Appl. No.: 10/832,043

(22) Filed: Apr. 26, 2004

(30) Foreign Application Priority Data
May 13, 2003 (FR) 03 05 728

(51) Int. Cl. A61F 2/04

(52) U.S. Cl. 623/23.64; 623/23.66; 606/151

(57) ABSTRACT

Device for fitting a uterine suspension prosthesis for the treatment of uterine prolapse, characterised in that it is constituted:

on the one hand, by a tubular trocar (1) which is provided at its proximal end with a lateral connector (5) capable of being connected to perfusion members, and at its distal end with a set of perforations (7) enabling a perfusion liquid to be evacuated to the outside, and

on the other hand, by a supple spindle (2) introduced into the internal portion of the tubular trocar (1) and provided at its proximal end with an eye (4) enabling the prosthesis to be slid, and at its distal end with an ogival rounded end-piece (3) constituting a guide member.

The present invention relates to a device for fitting a uterine suspension prosthesis for the treatment of uterine prolapse.

Regardless of its weight in the course of reproductive life or pregnancy, the uterus cannot normally fall into the vagina and is constantly held in a raised position by an assembly of connective fibres which are located behind the peritoneum and which ensure the positioning of the uterus in the manometric enclosure of the abdomen.

The connective fibres form two ligamentous bundles, namely a bundle of ligaments extending from the pubis to the sacrum and called the pubic genital sacral ligaments, and a bundle of oblique ligaments directed from the rear to the front and called the pelvic transversal and sagital hypogastric connective tissues.

Owing to the particular geometry of the peritoneal cavity in the region of its lower portion or neck, the uterus is located in its normal raised position above pressure pincers defined by the posterior portion of the peritoneum (pouch of Douglas) and the anterior portion of the peritoneum (utero-vesical pouch) located between the uterus and the bladder.

In the presence of abdominal efforts, those two pouches close on the conical funnel of the neck of the uterus, so that the uterus is pushed back up and cannot fall into the vagina.

However, it is relatively common for the ligaments that support the uterus, in particular the lateral connective fibres of the pelvic transversal and sagital hypogastric connective tissues, to slacken and deteriorate with pregnancy or age; such a deterioration may lead to a descent of the uterus out of the manometric enclosure of the abdomen; it is then expelled into the vagina every time pressure occurs. Uterine prolapse causes functional discomfort which may manifest itself only during intense efforts, or which may be permanent, and may therefore have a greatly incapacitating character.

Specialists propose various means of treating uterine prolapse which consist, in particular, in securing the uterus to the vertebral column, closing the pouch of Douglas, and even removing the uterus.

However, none of those means is of an entirely satisfactory nature, as demonstrated by the multiplicity of techniques that have been advocated for a long time now.

The idea on which the invention is based consisted in fitting between the peritoneum and the abdominal wall a prosthesis constituted by a strip of synthetic meshwork and in securing that strip, without stress, on the one hand, to the neck of the uterus by its lower portion and, on the other hand, to the abdominal aponeuroses by its upper portion, as a replacement for defective connective fibres.

Such a prosthesis enables uterine prolapse to be corrected satisfactorily if it puts the uterus back above the pressure pincers of the pouch of Douglas, which it is important to respect, and therefore into the manometric enclosure of the abdomen.

In addition, even though it has to be carried out under general anaesthesia, the fitting thereof is a distinctly less serious operation than the operations conventionally carried out to correct uterine prolapse.

The object of the invention is to propose a device permitting the fitting of such a uterine suspension prosthesis.

According to the invention, this device is characterised in that it is constituted, on the one hand, by a tubular trocar, particularly of metal, and, on the other hand, by a spindle of supple plastics material introduced into the internal portion of the tubular trocar and projecting at the lower portion thereof.

The tubular trocar is provided at a first of its ends, or its proximal end, with a lateral connector of the Luer type which is capable of being connected to perfusion members, and at its second end, or distal end, with a set of sprinkler-rose-type perforations extending over a length of approximately 1 cm and permitting the evacuation to the outside of a perfusion liquid, in particular physiological serum.

The spindle is for its part provided at its proximal end with an eye enabling the prosthesis to be slid, and at its distal end with an ogival rounded end-piece, preferably of foam, constituting a guide member.

According to a particularly advantageous feature of the invention, the spindle has a cross-section corresponding to the internal cross-section of the tubular trocar and is equipped on its periphery with at least one longitudinal groove constituting an irrigation channel permitting the transfer of the perfusion liquid, in particular physiological serum, from the top to the bottom towards the distal end of the trocar in order to permit the evacuation thereof to the outside.

It should be noted that, without departing from the scope of the invention, the diameter of the spindle may also be distinctly smaller than the internal cross-section of the trocar in order to permit the passage of the irrigation liquid.

The fitting of the prosthesis requires the perforation of the patient's abdomen, at the upper portion, and the introduction of the ogival distal end of the spindle by way of the orifice thus created.

The practitioner must then move the trocar/spindle assembly between the peritoneum and the abdominal wall, causing it to progress towards the bottom and towards the front of the abdomen as far as the neck of the uterus, passing behind the bladder.

In order to facilitate that progression of the trocar/spindle assembly at the internal portion of the peritoneal cavity, the trocar preferably has a double concavity in the space between its proximal end and its distal end, so that it is first of all concave on the inside and at the same time slightly concave towards the front.

The length of the device according to the invention depends for its part largely on the size and the corpulence of the patients.

In the course of the progression of the device in the peritoneal cavity, the connector of the trocar is constantly connected to a perfusion pouch containing physiological serum.

Consequently, physiological serum is constantly being evacuated to the outside in the immediate vicinity of the distal end equipped with a sprinkler-type rose.

The function of the serum is to enable the spaces to be opened by detaching the peritoneum from the abdominal wall and also from the iliac vessels, the ureter and the bladder, in order to permit access, behind the bladder, to the neck of the uterus to be supported.

More precisely, in the course of its progression, the position of the end of the spindle is located by coelioscopy or by echography under the peritoneum which moves away from the abdominal wall and the viscera (vessels, uterus, bladder) owing to the slackening caused by the passage under low pressure (one metre of water) of the physiological serum.

The spindle is thus guided under visual control under the round ligament, then between the uterus and the bladder in front of the uterine isthmus.

Before introducing the device, the surgeon will have prepared a pericervical incision by natural routes and will have slightly detached the lower portion of the junction between the bladder and the uterine isthmus.

When the progression is complete, the end of the spindle is thus near the surgeon's finger which has been introduced into the vagina and which guides the passage thereof so that it is free in the vagina.

In order to fit the prosthesis which is inserted into the eye provided at the proximal end of the spindle, it is then necessary merely to exert a downward traction on the ogival end of the spindle projecting at the proximal end of the metal trocar.

It then only remains for the practitioner to secure the prosthesis, on the one hand, to the neck of the uterus by its lower portion and, on the other hand, to the abdominal aponeuroses by its upper portion.

According to another feature of the invention, the tubular trocar may be provided with a support handle at its proximal end, in the vicinity of the lateral connector.

It should be noted that it is absolutely necessary to provide for each intervention two symmetrical devices, one of which is adapted to the right-hand side while the other is adapted to the left-hand side.

The features of the device forming the subject-matter of the invention will be described in greater detail with reference to the appended non-limiting drawings in which:

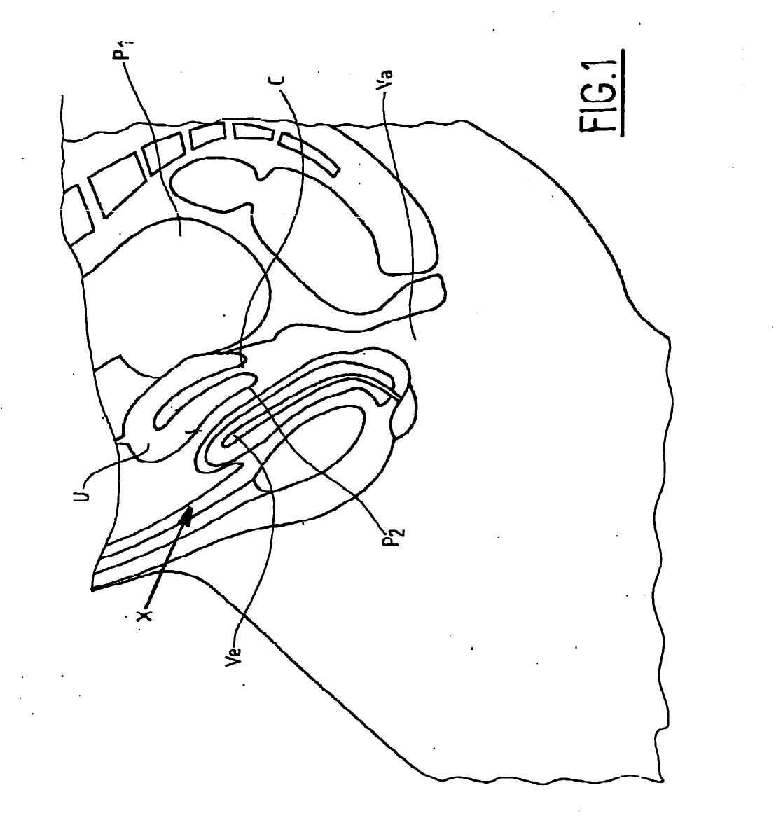

FIG. 1 is a diagrammatic section representing the female genital organs;

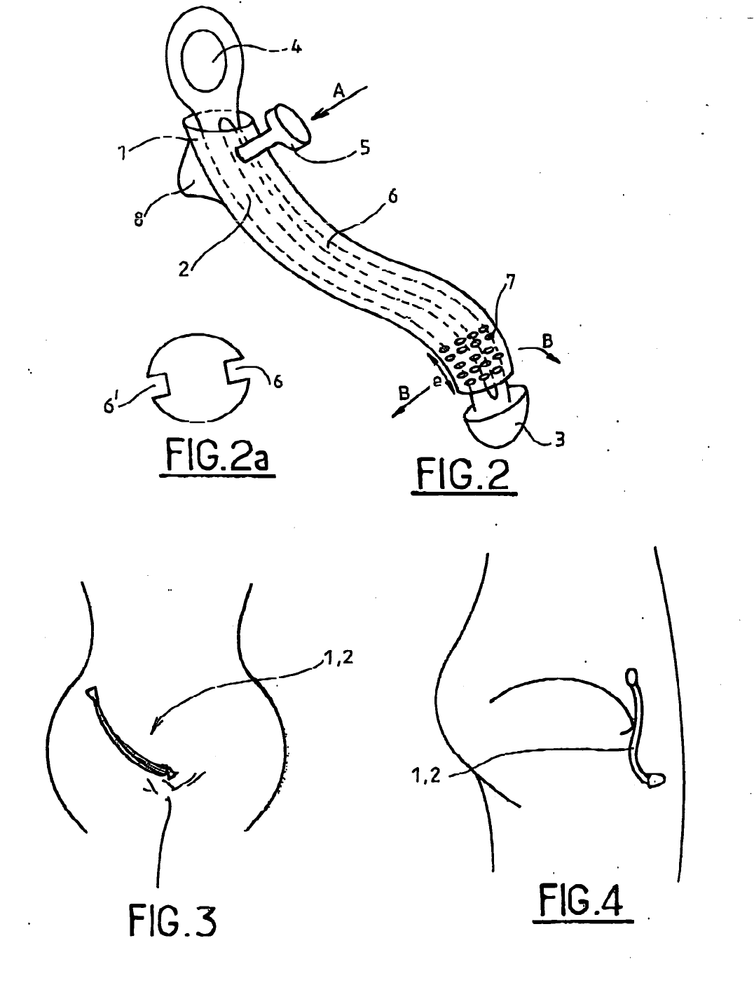

FIG. 2 is a perspective view of the device;

FIG. 2a is a cross-section of the spindle;

FIGS. 3 and 4 are diagrammatic front and side views, respectively, of a patient showing the implantation of the device.

According to FIG. 1, the uterus U cannot normally fall into the vagina Va irrespective of its weight, even in the course of pregnancy, and it is held in a raised position by the abdominal pressure.

In the presence of an abdominal effort, the posterior portion of the peritoneum P₁ forming the pouch of Douglas and the anterior portion of the peritoneum P₂ forming the utero-vesical pouch approach one another from each side of the neck C of the uterus in such a manner as to push the uterus U back up.

However, the ligamentous bundles (not shown) which hold the uterus in the raised position may deteriorate so that the uterus U falls into the vagina Va and may thus cause considerable discomfort.

That constitutes uterine prolapse.

The function of the device shown in FIGS. 2 to 4 is to enable the prolapse to be treated by maintaining the uterus in the abdomen without traction.

According to FIG. 2, the device is constituted by a tubular metal trocar 1 which has a circular cross-section and a length of the order of 20 cm and into which is introduced a spindle 2 of supple plastics material which has a corresponding circular cross-section and which projects from each end of the trocar 1.

The spindle 2 is provided at its distal end with an ogival rounded end-piece 3 of foam which constitutes a guide member.

The spindle 2 is also provided at its proximal end with an eye 4 which is to receive a prosthesis (not shown) constituted by a strip of synthetic meshwork.

The trocar 1 is for its part provided at its proximal end with a lateral connector 5 of the Luer type which is to be connected to a perfusion pouch in order to enable a stream of physiological serum shown diagrammatically by the arrow A to be introduced into the internal portion of the trocar, and also, where appropriate, with a support handle 8.

According to FIGS. 2 and 2a two longitudinal grooves 6, 6′ constituting two irrigation channels are provided on the external periphery of the spindle 2.

The physiological serum coming from the perfusion members can thus flow inside the trocar 1 along those two longitudinal grooves 6, 6′ as far as the distal end of the device.

According to FIG. 2, the trocar 1 is provided in that region with a set of sprinkler-rose-type perforations 7 extending over a length e of approximately 1 cm and via which the physiological serum can be evacuated to the outside in accordance with the arrows B.

According to FIGS. 1, 3 and 4, in order to fit the prosthesis (not shown), the practitioner has to perforate the upper portion of the patient's abdomen, introduce the device 1, 2 into the orifice thus created in accordance with the arrow X (FIG. 1) and move it from the top to the bottom between the peritoneum and the abdominal wall, passing behind the bladder Ve, as far as the neck C of the uterus.

In order to permit that progression, and as shown more precisely in FIGS. 3 and 4, the trocar 1 has a double concavity in the space, on the inside (FIG. 3) and towards the front (FIG. 4).

When the ogival distal end 3 of the spindle 2 has reached the neck of the uterus, the practitioner, who has previously made a perforation and performed the cleavage between the uterine isthmus and the bladder, grasps the device 1, 2 manually in order to introduce it into the vagina before exerting a traction thereon in such a manner as to enable the prosthesis (meshwork) engaged in the upper eye of the supple spindle to be pulled downwards.

It then only remains for him to secure the meshwork, on the one hand, to the neck of the uterus by its lower portion and, on the other hand, to the abdominal aponeuroses by its upper portion.

1. Device for fitting a uterine suspension prosthesis for the treatment of uterine prolapse, the prosthesis being constituted by a strip of synthetic meshwork which is to be fitted between the peritoneum and the abdominal wall and secured, on the one hand, to the neck of the uterus by its lower portion and, on the other hand, to the abdominal aponeuroses by its upper portion, as a replacement for defective connective fibres, characterised in that it is constituted:
on the one hand, by a tubular trocar (1), particularly of metal, which is provided at a first of its ends, or its proximal end, with a lateral connector (5) of the Luer type capable of being connected to perfusion members, and at its second end, or distal end, with a set of sprinkler-rose-type perforations (7) extending over a length of approximately 1 cm and enabling a perfusion liquid, in particular physiological serum, to be evacuated to the outside, and
on the other hand, by a spindle (2) of supple plastics material introduced into the internal portion of the tubular trocar (1) and projecting from each end thereof, the spindle (2) being provided at its proximal end with an eye (4) enabling the prosthesis to be slid, and at its distal end with an ogival rounded end-piece (3), preferably of foam, constituting a guide member.

2. Device according to claim 1, characterised in that the spindle (2) has a cross-section corresponding to the internal cross-section of the tubular trocar (1) and is equipped on its periphery with at least one longitudinal groove (6, 6′) constituting an irrigation channel permitting the transfer of the perfusion liquid, in particular physiological serum, from the top to the bottom towards the distal end of the trocar (1) in order to permit the evacuation thereof to the outside.

3. Device according to claim 1, characterised in that the diameter of the spindle is distinctly smaller than the internal cross-section of the trocar in order to permit the passage of the perfusion liquid, in particular physiological serum.

4. Device according to claim 1, characterised in that the tubular trocar (1) has a double concavity in the space between its proximal end and its distal end in order to enable it to be directed towards the bottom and towards the front of the abdomen.

5. Device according to claim 1, characterised in that the tubular trocar (1) is provided with a support handle (8) at its proximal end, in the vicinity of the lateral connector (5).
